(19) United States
(12) Patent Application Publication
(10) Pub. No.: US 2008/0119722 A1
(43) Pub. Date: May 22, 2008

(54) BODY COMPRESSION DEVICE FOR MEDICAL IMAGING

(76) Inventor: Charles M. Swaney, Columia, MO (US)

Correspondence Address: LATHROP & GAGE LC, 2345 GRAND AVENUE, SUITE 2800, KANSAS CITY, MO, 64108, US

(21) Appl. No.: 11/562,543

(22) Filed: Nov. 22, 2006

(51) Int. Cl. A61B 5/055; A61B 6/10

(52) U.S. Cl. 600/415; 128/846

(57) ABSTRACT

A body compression device for medical imaging of a patient includes a pad that wraps about the patient and is secured about the patient, reshaping adipose tissue of the patient to facilitate the medical imaging. Another body compression device for medical imaging includes dorsal and ventral panels, and elastic side panels affixed to one or both of the dorsal and ventral panels. The dorsal and ventral panels are secured about the patient. A method of reducing total radiation exposure during CT imaging includes an X-ray fiducial device embedded in the ventral panel. A CT pre-scan image of the patient is generated. A medical region of interest of the patient is determined from a position of the device in the CT pre-scan image. Radiation shielding is placed on one or more portions of the patient that are outside the medical region of interest before the CT imaging is performed.

### BACKGROUND

Adipose tissue creates a unique set of challenges in computerized tomography (“CT”) imaging of patients. For example, CT imaging of a patient is affected when there is a large amount of adipose tissue below the skin of the patient's body; such tissue may settle in such a way as to cause imaging artifacts and/or require increased radiation levels to generate a satisfactory image. Increased X-ray radiation is disadvantageous, as it poses a risk of damage to and/or cancer of certain body tissues. Breasts that sag laterally when a patient is in a supine position can generate similar challenges, furthermore, breasts can be radiosensitive; that is, breast tissue may have a greater predisposition than other tissue for damage and/or cancer from CT imaging X-rays. Current trends toward obesity in the human population exacerbate these imaging challenges and risks.

FIG. 1 (prior art) shows a schematic side view of a CT system 10 with a patient 20(1) lying supine on a gurney 30. A patient surface 35 of a gurney 30, bearing patient 20(1) being imaged, moves in and out of CT system 10 in the direction of an arrow 40.

FIG. 2 (prior art) shows a schematic front view of CT system 10 with an obese patient 20(2) lying supine on gurney 30. A gantry 15 of CT system 10 forms a space 17. As gurney 30 moves at least a portion of patient 20(2) within space 17, one or more X-ray sources (not shown) within gantry 15 emit X-rays that interact with tissues of patient 20(2) and are detected by sensors (not shown) within gantry 15. CT system 10 utilizes signals from the sensors to reconstruct images of the tissues. Adipose tissue of patient 20(2) settles downwards and laterally, so that a cross-section of patient 20(2) resembles a trapezoid in the region shown at A, instead of the circular or ellipsoidal profile formed by the body of a normal weight patient. Sides of patient 20(2) may also touch gantry 15 of CT system 10 as patient 20(2) moves within space 17, causing patient discomfort and/or artifacts in images produced by system 10. Furthermore, increased X-ray radiation (as compared to X-ray radiation used to image a normal weight patient) may be required to penetrate the adipose tissue in order to produce satisfactory CT images. The CT imaging industry has attempted to address the issues outlined above by developing CT systems with larger gantry openings; however, larger gantry diameters increase the cost of a CT system, and do not reduce the radiation needed to produce satisfactory images.

### SUMMARY

In one embodiment, a body compression device for medical imaging of a patient includes a pad configured to wrap about the patient, and means for securing the pad about the patient. Adipose tissue of the patient is reshaped to facilitate the medical imaging when the pad is secured about the patient.

In one embodiment, a body compression device for medical imaging of a patient includes a pad configured to wrap about the patient, the pad having first and second ends; and at least one keeper to secure the first end of the pad to the second end of the pad about the patient. Adipose tissue of the patient is reshaped to facilitate the medical imaging when the pad is secured about the patient.

In one embodiment, a body compression device for medical imaging of a patient includes a dorsal panel, a ventral panel, elastic side panels affixed to one or both of the dorsal and ventral panels; and means for securing the dorsal and ventral panels about the patient. Adipose tissue of the patient is reshaped to facilitate the medical imaging when the pad is secured about the patient.

In one embodiment, a method of providing a reference as to the proper positioning of external radiation shielding during CT imaging includes securing an X-ray fiducial marker to the body compression device or positioning the fiducial marker upon the body compression device. A CT pre-scan image of the patient is generated. A medical region of interest of the patient is determined from a position of the device in the CT pre-scan image. Radiation shielding is placed on one or more portions of the patient that are outside the medical region of interest before the CT imaging is performed.

In one embodiment, a method of medical imaging of a patient includes positioning a substantially radiolucent body compression device adjacent to the patient. the body compression device is secured about the patient, thereby reshaping adipose tissue of the patient to reduce a horizontal diameter of the patient's cross-section. The patient is subsequently imaged.

### BRIEF DESCRIPTION OF DRAWINGS

The present disclosure may be understood by reference to the following detailed description taken in conjunction with the drawings briefly described below. It is noted that, for purposes of illustrative clarity, certain elements in the drawings may not be drawn to scale. Specific instances of an item may be referred to by use of a numeral in parentheses (e.g., buckle end 16(1)) while numerals without parentheses refer to any such item (e.g., buckle ends 16).

FIG. 1 shows an elevation view of a average body fat composition patient positioned atop a gurney on a surface and being readied for movement into a CT system.

FIG. 2 shows a perspective view of high body fact composition patient positioned atop the CT system gurney and ready for entry into the CT system.

FIG. 3 shows a distal side of a body compression device for medical imaging of a patient, in accord with an embodiment.

FIG. 4 shows a proximal side of the body compression device of FIG. 3.

FIG. 5 shows the body compression device of FIG. 3 fastened about a patient in preparation for imaging by a CT system.

FIG. 6A and FIG. 6B show a proximal view and a cross-section, respectively, of a body compression device for medical imaging of a patient, in accord with an embodiment.

FIG. 7A and FIG. 7B show a proximal view and a cross-section, respectively, of a body compression device for medical imaging of a patient, in accord with an embodiment.

FIGS. 8A and 8B show distal sides and a cross-section, respectively, of sections that form a body compression device for medical imaging of a patient.

### DETAILED DESCRIPTION OF DRAWINGS

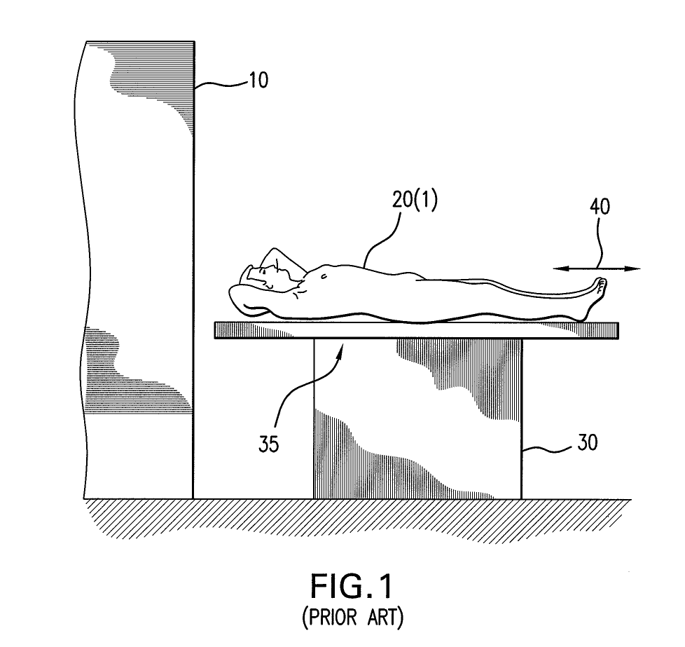

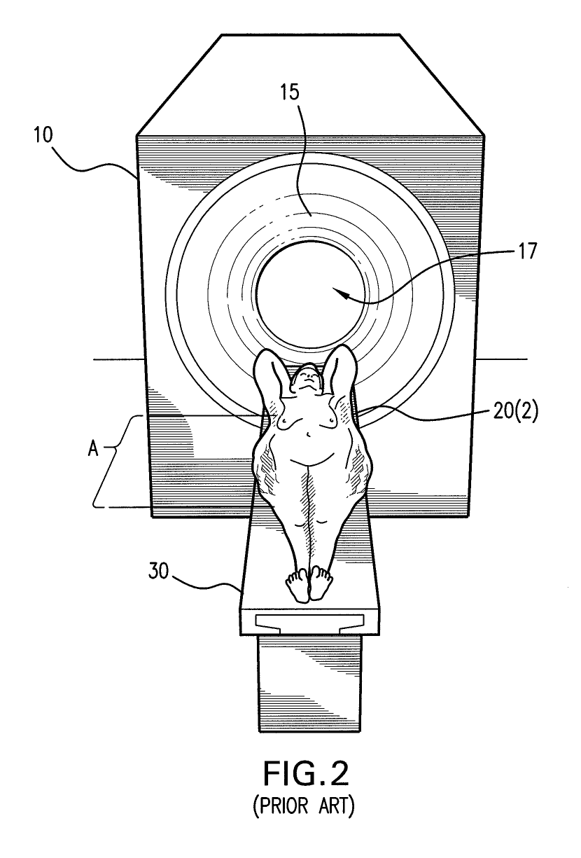

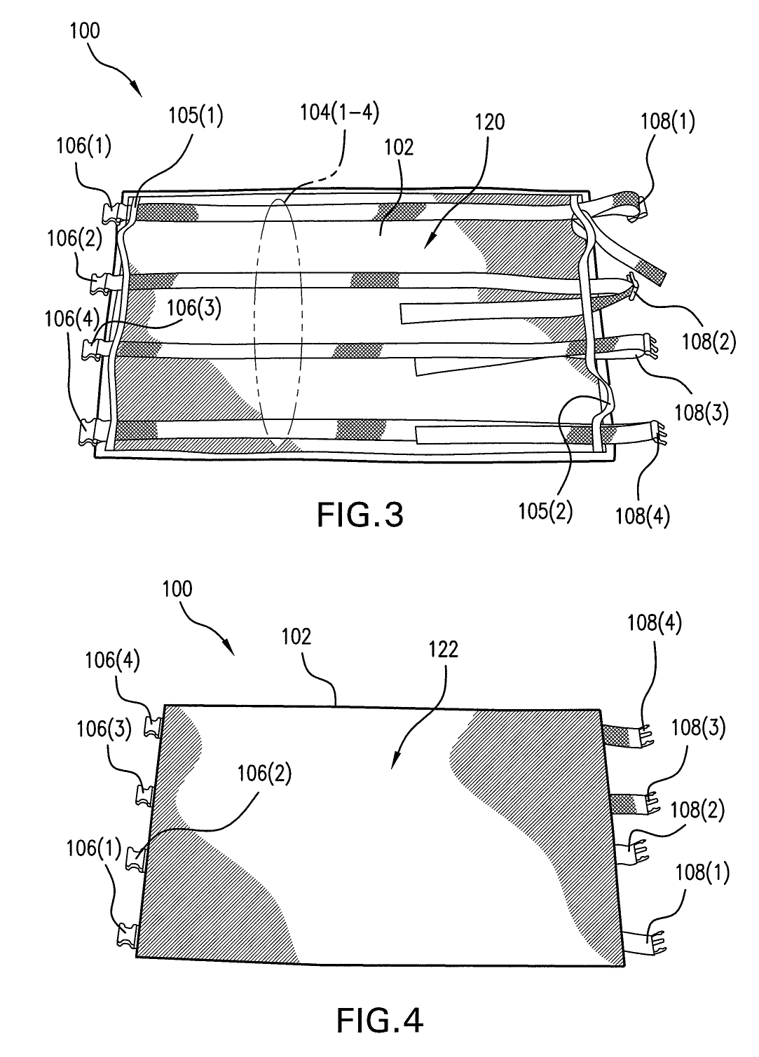

FIG. 1 depicts an average body fat composition patient 20(1) in position atop a CT system gurney 30 revealing the direction of motion 40 of the gurney platform. FIG. 2 reveals a high body fat composition patient 20(2) ready for entry into the space 17 of a CT system 10. Segment A of FIG. 2 shows the increased circumference area of the high body fat composition patient 20(2) that often times interferes with the structural components that define the space 17 of the gantry 15. FIG. 3 shows a body compression device 100 for medical imaging of a patient (e.g., patient 20(2)). Device 100 includes a pad 102 and four straps 104(1)-104(4). Each of straps 104 connects with a corresponding one of female buckle ends 106(1)-106(4) and a corresponding one of male buckle ends 108(1)-108(4). Straps 104 are held loosely in place on pad 102 by holding straps 105(1) and 105(2). One or more of buckle ends 106 and 108 may include means for adjusting tightness of straps 104. In FIG. 3, a distal side 120 of device 100 (that is, a side of pad 102 that will face away from a patient when in use) faces upwards.

FIG. 4 shows body compression device 100 with a proximal side 122 of pad 102 facing upwards.

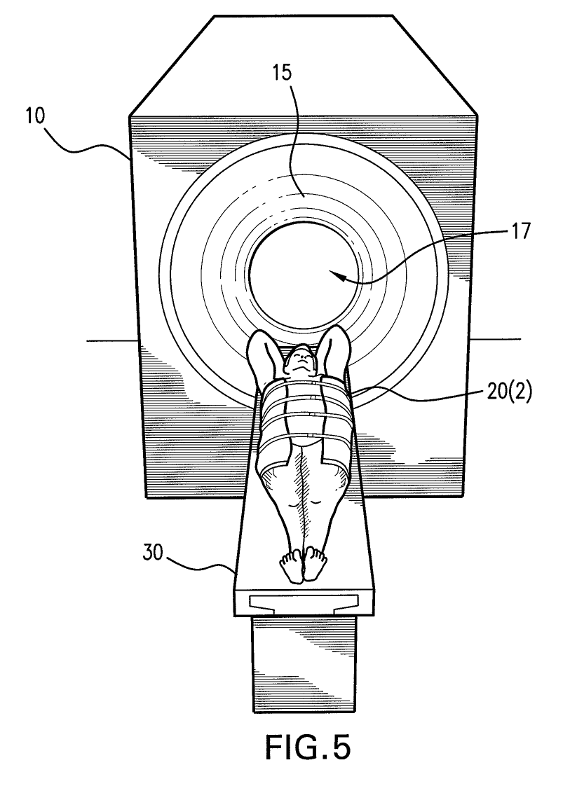

When body compression device 100 is used in CT imaging, device 100 is placed on a gurney of a CT imaging system with proximal side 122 facing upwards, and with straps 104 oriented perpendicular to a direction of travel of a gurney patient surface into a CT system (e.g., straps 104 would be horizontal, in the perspective shown in FIG. 2; see also FIG. 5). A patient lies on device 100; a nurse or CT technician wraps the ends of pad 102 around the patient's sides, fastens each buckle end 108(1)-108(4) into the corresponding buckle end 106(1)-106(4) and adjusts tightness of each strap. In this manner, adipose tissues of the patient that would otherwise sag are reshaped into an ellipsoidal shape that, in cross section, resembles the cross-section of a normal weight patient. The gurney moves into the gantry of the CT system, and imaging proceeds as for a normal weight patient. The body shape produced by use of body compression device 100 about an obese patient facilitates improved CT imaging, as compared to imaging of the patient without body compression device 100. The improvement arises because (a) body tissues that might otherwise sag towards the gantry of the CT system are kept further away from the gantry, eliminating certain image artifacts, and (b) a horizontal diameter of the patient's cross-section is reduced, reducing a thickness through which imaging X-rays must pass, so that radiation utilized by the system for imaging can be correspondingly reduced while producing satisfactory images.

FIG. 5 shows body compression device 100 fastened about patient 20(2) in preparation for imaging by CT system 10. As shown, body compression device 100 reshapes body tissues of patient 20(2) into an ellipsoidal shape, as opposed to the generally trapezoidal shape shown at A in FIG. 2.

Pad 102 may be formed of two layers of, for example, vinyl with padding (e.g., ¼″ thick polyurethane foam, not shown) between the layers. Other materials may also be used to form pad 102; preferable materials are radiolucent, flexible (but not highly elastic) and impermeable to water and other bodily fluids. Flexibility without elasticity promotes the ability of pad 102 to conform to body tissues and hold them in place; waterproof and impermeable materials make pad 102 easy to clean, and resist picking up stains and odors. Pad 102 may include fasteners (e.g., buttons, snaps or zippers, adhesive fabrics such as VELCRO™, or even simple folds of the layers of pad 102) that hold padding in place but allow its removal for cleaning or replacement. Straps 104 and 105 are preferably radiolucent, water impermeable, durable, wide enough to hold necessary weight (e.g., in the range of tens of pounds per strap 104) and capable of being sewn with heavy-duty sewing machines. Straps 104 may be loosely held in position on pad 102 using holding straps 105(1), 105(2) as shown in FIG. 3. Straps 105 may also be utilized as handles to carry pad 102 and/or to assist in positioning pad 102 about a patient. Alternatively, straps 104 may be sewn directly to pad 102, or may connect with pad 102 using other fasteners (e.g., snaps). Buckle ends 106 and 108 are preferably radiolucent, water impermeable, durable and strong enough to hold the necessary weight.

Other changes may be made in body compression device 100 without departing from the scope hereof. For example, although body compression device 100 is shown with four straps 104, more or fewer straps may be utilized. The buckles shown are one example of a “keeper” that secures one end of each strap 104 to another end of each strap 104 around patient 20(2). Other fasteners such as buttons, snaps, zippers or adhesive fabrics (e.g., hook-and-loop fastening fabrics such as VELCRO™) may also be utilized as a keeper. Furthermore, instead of configuring straps 104 as a keeper, opposing edges of pad 102 may be configured as a keeper by utilizing securing means (e.g., snaps, buttons, buckles, zippers, adhesive fabrics) directly integrated with pad 102 to secure compression device 100 about a patient, so that straps are not needed. Such securing means are preferably strong (enough to securely hold the weight of a patient's adipose tissue), radiolucent and impermeable to bodily fluids. Radiation shielding (e.g., lead) may be integrated into portions of compression device 100 to minimize patient exposure to X-rays at locations not being imaged, and pad 102 may include fasteners (e.g., buttons, snaps or zippers, adhesive fabrics, or folds) that hold the shielding in place but allow its removal and/or adjustment of its position. Compression device 100 may also be utilized to reshape tissues of patients for other types of imaging, such as magnetic resonance imaging.

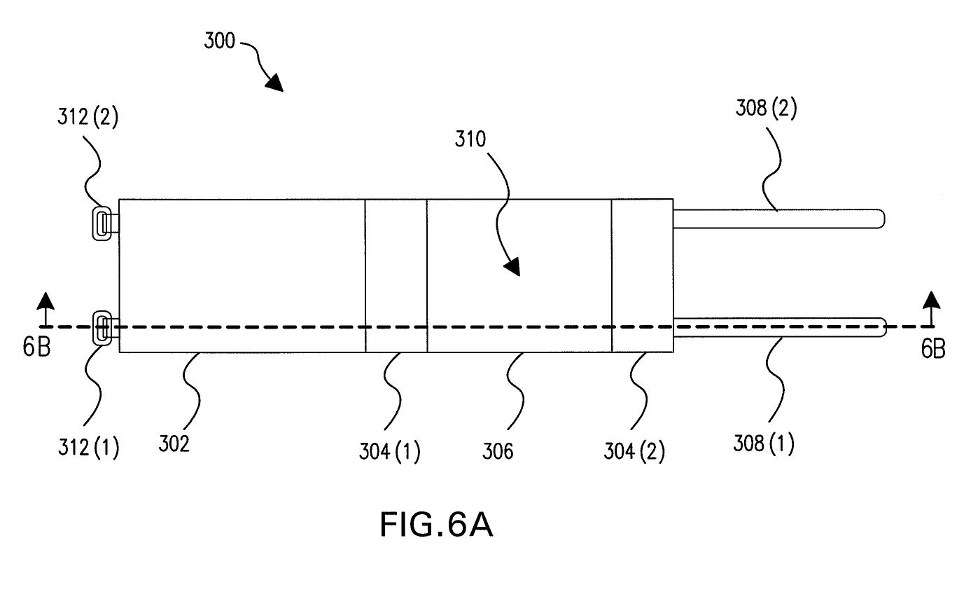

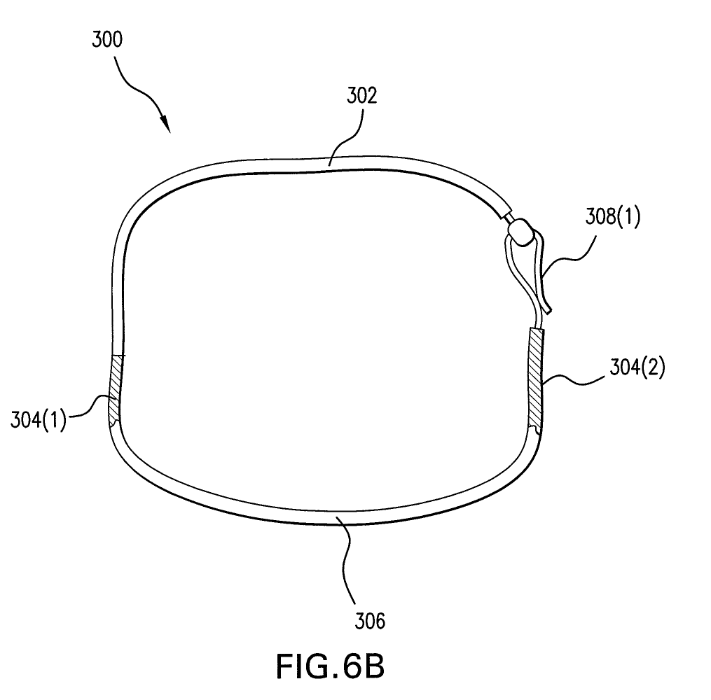

FIG. 6A and FIG. 6B show a proximal view and a cross-section, respectively, of a body compression device 300 for medical imaging of a patient. Body compression device 300 may be utilized, for example, to facilitate CT imaging of patients with breasts that sag outward when the patient's body is in a supine position. Body compression device 300 is shown with a proximal side 310 facing upwards, and includes a ventral panel 302, two side panels 304(1) and 304(2), and a dorsal panel 306, forming a contiguous unit. Straps 308(1) and 308(2) attach to side panel 304(2). Ventral panel 302 and dorsal panel 306 may be made of similar materials as pad 102 of body compression device 100, however, ventral panel 302 may alternatively be made of somewhat elastic material for added patient comfort. Straps 308 may be made out of similar materials as straps 104 and 105 of device 100, and loops 312 may be made out of similar materials as buckle ends 106 and 108 of device 100. Side panels 304 are preferably formed of materials that are flexible, radiolucent and impermeable, and are somewhat elastic, to facilitate patient comfort in breathing.

In use, a patient lies supine on proximal side 310 of dorsal panel 306; ventral panel 302 wraps over the patient's chest. The patient and/or a nurse or CT technician cooperate to displace the patient's breasts upward, then thread each of straps 308(1) and 308(2) through a corresponding one of loops 312(1) and 312(2) and tighten each strap 308. Each strap 308 doubles back on itself, and fastens with adhesive fabric, forming a keeper that secures the ends of device 300 together. Ventral panel 302 holds the patient's breasts upward, so that they remain largely out of the path of imaging X-rays. FIG. 6B shows a cross-section of body compression device 300 along the line marked in FIG. 6A, and in a shape formed when device 300 is fastened about a patient's body. It is contemplated that the arrangement of straps 308 and loops 312 may be reversed, with straps 308 attached to ventral pad 302 and loops 312 attached to side panel 304(2), rather than the arrangement shown.

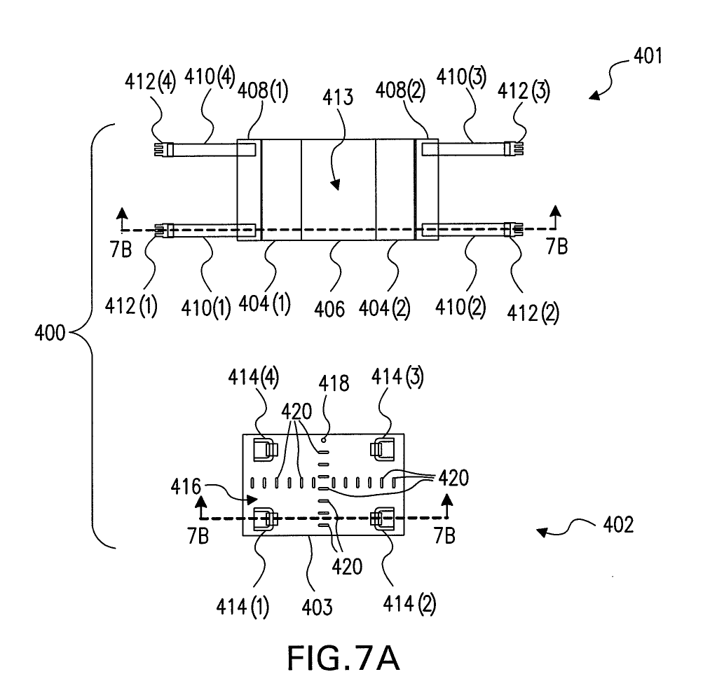

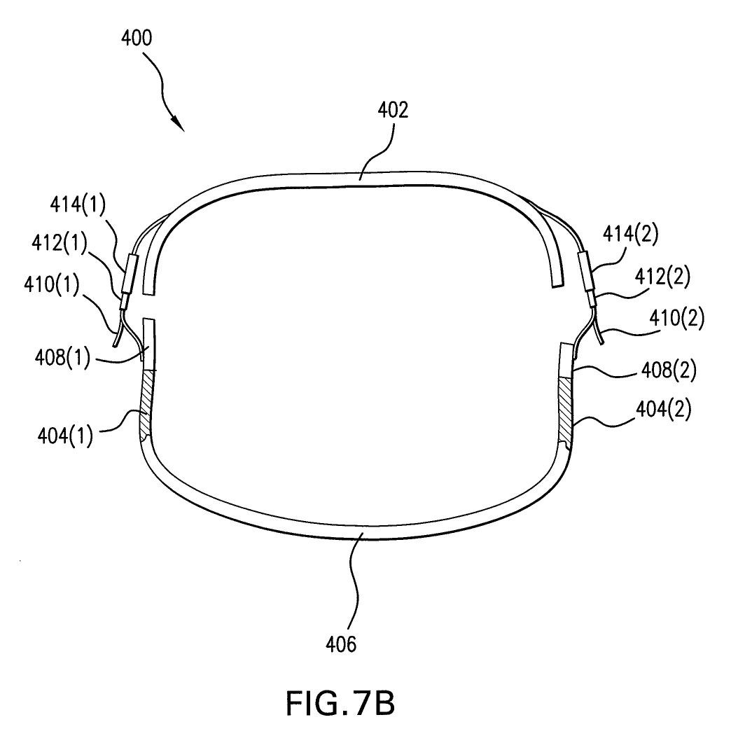

FIGS. 7A and 7B show a proximal view and a cross-section, respectively, of a body compression device 400 for medical imaging of a patient. Body compression device 400 may be utilized, for example, to facilitate CT imaging of patients with breasts that sag outward when the patient's body is in a supine position. FIG. 7A shows body compression device 400 as separate sections 401 and 402. Section 401 is shown with a distal side 413 facing upwards, and includes a dorsal panel 406, two elastic side panels 404(1) and 404(2), two inelastic side panels 408(1) and 408(2), and straps 410(1)-410(4), each of which connects with a corresponding one of buckle ends 412(1)-412(4). Section 402 is shown with a distal side 416 facing upwards, and includes a ventral panel 403 having buckle ends 414(1)-414(4) mounted thereto. Ventral panel 403 also includes an X-ray fiducial device 418 and visual markers 420 (some visual markers 420 are shown but not labeled in FIG. 7B, for clarity of illustration).

Ventral panel 403, dorsal panel 406 and inelastic side panels 408(1) and 408(2) may be made of similar materials as pad 102 of body compression device 100, however, ventral panel 403 may alternatively be made of somewhat elastic material for added patient comfort. Straps 410 may be made out of similar materials as straps 104 and 105 of device 100, and buckle ends 412 and 414 may be made out of similar materials as buckle ends 106 and 108 of device 100. Buckle ends 412 and 414, together, form keepers for securing sections 401 and 402 of device 400 about a patient. Buckle ends 412 and 414, or both, may include means for adjusting tightness of straps 410. Side panels 404 are preferably formed of materials that are flexible, radiolucent and impermeable, and are somewhat elastic, to facilitate patient comfort in breathing. X-ray fiducial device 418 is visible to a human, and is made of a material (e.g., metal) that is radiopaque and is preferably large enough to be seen on an X-ray image, but small enough that device 418 does not interfere with CT imaging of a patient. As resolution of currently available CT imaging systems is on the order of 1 millimeter, a practical size for device 418 is a diameter of 2 to 3 millimeters (e.g., about the size of BB shot). Visual markers 420 are preferably radiolucent and may be painted, stitched or otherwise formed on the surface of ventral panel 403 at known spacing from each other and from fiducial device 418. Visual markers 420 are given the appearance of short lines in FIG. 7A, but may be shaped differently, in particular they may form grid lines across body compression device 400.

In use, a patient lies supine on dorsal panel 406, and ventral panel 402 is placed over the patient's chest with distal side 416 facing upwards. The patient and/or a nurse or CT technician cooperate to displace the patient's breasts upward, then buckle together corresponding buckle ends 412 and 414, and adjust tightness of straps 410 as necessary. Ventral panel 403 holds the patient's breasts upward, so that they remain largely out of the path of imaging X-rays. FIG. 7B shows a cross-section of body compression device 400 along the line marked in FIG. 7A, and in a shape formed when device 400 is fastened about a patient's body.

A common practice in CT imaging is to perform a “pre-scan” with a CT scanner operating at a very low X-ray dose to generate a two-dimensional image (e.g., like a conventional X-ray image) to register a medical region of interest to a corresponding position of the CT scanner gurney. Then, a subsequent (cross-sectional) CT scan is confined to the region of interest. While this practice helps to reduce a total X-ray dose received by a patient, it may be difficult to register the position of the medical region of interest to external locations on the patient's body with precision. However, fiducial device 418, because it is both visible and radiopaque, enables a CT technologist to utilize a pre-scan image to register position of the medical region of interest to external locations on the patient's body with high accuracy. Unlike loose-fitting clothing and hospital gowns, a body compression device that includes fiducial device 418 secures a position of device 418 on a patient's body with high precision over an amount of time required to do a CT pre-scan and to prepare the system for a three dimensional CT scan. Visual markers 420 may further assist in determining the exact region of interest, since their location relative to fiducial device 418 is known. Given the information provided by fiducial device 418 and/or visual markers 420, a CT technician may provide a patient with additional shielding about parts of the body that are not being imaged, to further reduce X-ray exposure of such parts without detriment to the CT image.

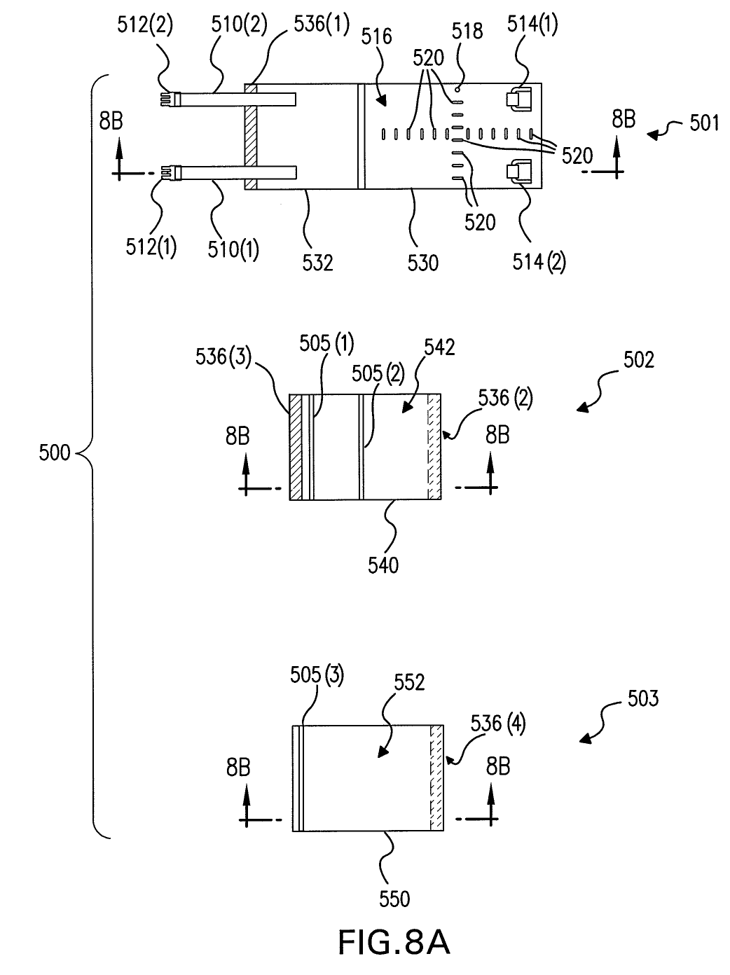

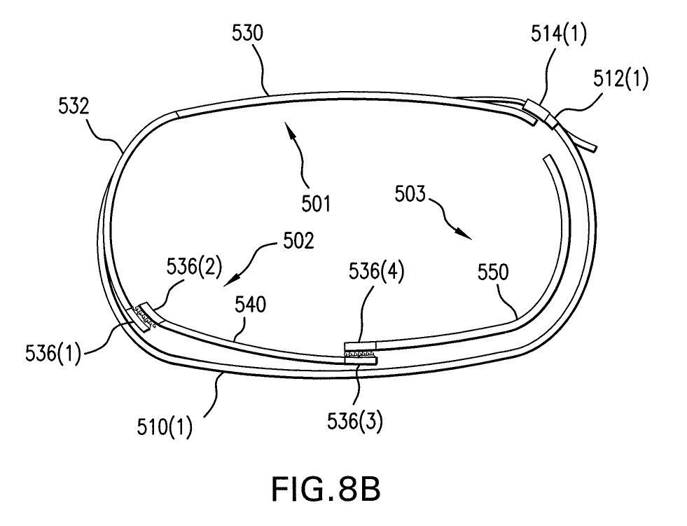

FIGS. 8A and 8B show distal sides and a cross-section, respectively, of sections 501, 502 and 503 that form a body compression device 500 for medical imaging of a patient. Body compression device 500 may be utilized, for example, to facilitate CT imaging of patients with breasts that sag outward when the patient's body is in a supine position.

FIG. 8A shows section 501 with a distal side 516 facing upwards. Section 501 includes a ventral panel 530, an elastic side panel 532 and an attaching side panel 536(1). Attaching side panel 536(1) includes a provision for attachment to an adjacent sections (e.g., sections 502 or 503, described below). Such provision for attachment may be a panel of adhesive fabric (e.g., VELCRO™) on distal side 516, or one part of a zipper. Ventral panel 530 includes an X-ray fiducial device 518 and visual markers 520 (some visual markers 520 are shown but not labeled in FIG. 8B, for clarity of illustration). Section 501 also includes straps 510(1), 510(2), each of which connects with a corresponding one of buckle ends 512(1), 512(2). Buckle ends 512 may include means for adjusting tightness of straps 510.

FIG. 8A shows section 502 with a distal side 542 facing upwards. Section 502 includes a panel 540 having holding straps 505(1), 505(2) mounted thereto. Holding straps 505 serve to position straps 510 when device 500 is placed around a patient. Section 502 further includes attaching side panels 536(2) and 536(3), which include provisions for attachment to adjacent sections (e.g., sections 501 or 503, described below). Such provision for attachment may be a panel of adhesive fabric (e.g., VELCRO™), or one part of a zipper. When attaching side panel 536(2) is a panel of adhesive fabric, such fabric is on a proximal side of section 502 (and is thus shown in dotted outline in FIG. 8A), so as to mate with attaching panel 536(1) of section 501. When attaching side panel 536(3) is a panel of adhesive fabric, such fabric is on a distal side of section 502, so as to mate with attaching panel 536(4) of section 503.

FIG. 8A shows section 503 with a distal side 552 facing upwards. Section 502 includes a panel 550 having holding strap 505(3) mounted thereto. Holding strap 505(3) serves to position straps 510 when device 500 is placed around a patient. Section 503 further includes an attaching side panels 536(4), which includes provision for attachment to an adjacent section (e.g., sections 501 or 502, described above). Such provision for attachment may be a panel of adhesive fabric (e.g., VELCRO™), or one part of a zipper. When attaching side panel 536(3) is a panel of adhesive fabric, such fabric is on a proximal side of section 502 (and is thus shown in dotted outline in FIG. 8A), so as to mate with panel 536(1) of section 501 or panel 536(3) of section 502.

Body compression device 500 thus exemplifies a modular system wherein sections 502 and/or 503 can be utilized in as many instances as needed to fit about a patient of any size. For example, a nurse or CT technician might fit a normal weight patient with sections 501 and 503, attaching panel 536(1) with panel 536(4), wrapping straps 510 about the patient and tightening straps 510 to fit the patient. Alternatively, in the case of a moderately obese patient, the nurse or CT technician might fit the patient with sections 501, 502 and 503, attaching panel 536(1) with panel 536(2) and panel 536(3) with panel 536(4), lengthening straps 510 to fit about the patient and tightening straps 510 when all of sections 501, 502 and 503 have been encircled about the patient. Alternatively, in the case of a moderately obese patient, the nurse or CT technician might fit the patient with sections 501, 502 and 503, attaching panel 536(1) with panel 536(2) and panel 536(3) with panel 536(4), lengthening straps 510 to fit about the patient and tightening straps 510 when all of sections 501, 502 and 503 have been encircled about the patient. Still alternatively, in the case of an extremely obese patient, the nurse or CT technician might fit the patient with sections 501, multiple sections 502, and 503, attaching panel 536(1) with panel 536(2) of a first section 502, attaching first panel 536(3) with panel 536(2) of a second section 502, attaching second panel 536(3) with panel 536(4). Straps 510 would be further lengthened to fit about the patient and would be tightened when all of sections 501, multiple sections 502, and 503 have been encircled about the patient.

Panels 530, 540 and 550 may be made of similar materials as pad 102 of body compression device 100, however, panel 530 may alternatively be made of somewhat elastic material for added patient comfort. Straps 510 may be made out of similar materials as straps 104 and 105 of device 100, and buckle ends 512 and 514 may be made out of similar materials as buckle ends 106 and 108 of device 100. Buckle ends 512 and 514, together, form keepers for securing sections 501, 502 and/or 503 of device 500 about a patient. Buckle ends 512 and 514, or both, may include means for adjusting tightness of straps 510. X-ray fiducial device 518 is visible to a human, and is made of a material (e.g., metal) that is radiopaque and is preferably large enough to be seen on an X-ray image, but small enough that device 518 does not interfere with CT imaging of a patient. As resolution of currently available CT imaging systems is on the order of 1 millimeter, a practical size for device 518 is a diameter of 2 to 3 millimeters (e.g., about the size of BB shot). Visual markers 520 are preferably radiolucent and may be painted, stitched or otherwise formed on the surface of ventral panel 503 at known spacing from each other and from fiducial device 518. Visual markers 520 are given the appearance of short lines in FIG. 8A, but may be shaped differently, in particular they may form grid lines across body compression device 500.

In use, a nurse or CT technician pre-assembles device 500 with section 501 and appropriate sections 502 and/or 503 based on the size of the patient. The patient lies supine on device 500, which is wrapped about the patient's chest. The patient and/or the nurse or CT technician cooperate to displace the patient's breasts upward, then buckle together corresponding buckle ends 512 and 514, and adjust tightness of straps 510 as necessary. Panel 530 holds the patient's breasts upward, so that they remain largely out of the path of imaging X-rays. FIG. 8B shows a cross-section of body compression device 500 along the line marked in FIG. 8A, and in a shape formed when device 500 is fastened about a patient's body.

The changes described above, and others, may be made in the body compression device described herein without departing from the scope hereof. For example, elements that are shown in specific embodiments herein may be utilized in other embodiments than those shown. Keepers that secure edges or parts of the body compression devices to each other may include substitution of elements such as buckles, loops, snaps, zippers, adhesive fabric and/or folds, for the particular elements shown. X-ray fiducial devices and/or visual markers may be utilized in any embodiment, and may differ in appearance from those shown. Elastic side panels may be affixed to dorsal and/or ventral pads of a body compression device in differing configurations from those shown. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

What is claimed is:

1. A body compression device for medical imaging of a patient, comprising:
a pad configured to wrap at least partly about the patient; and
means for securing the pad about the patient;
such that adipose tissue of the patient is reshaped to facilitate the medical imaging when the pad is secured about the patient.

2. Body compression device of claim 1, the pad extending from armpits of the patient to thighs of the patient.

3. Body compression device of claim 1, the pad comprising material that is radiolucent, waterproof and flexible.

4. Body compression device of claim 1, the pad comprising padding.

5. Body compression device of claim 4, the pad comprising means for alternately securing, and allowing removal of, the padding.

6. Body compression device of claim 1, the pad comprising means for securing radiation shielding to the pad.

7. Body compression device of claim 1, the means for securing comprising straps with buckles for alternately securing the pad about the patient and releasing the pad from the patient.

8. Body compression device of claim 1, the means for securing comprising adhesive fabric.

9. Body compression device of claim 1, the means for securing comprising one or more zippers.

10. Body compression device of claim 1, further comprising an X-ray fiducial device for registering a medical region of interest to external location on a patient's body.

11. Body compression device of claim 1, the pad forming a first section, the device additionally comprising a second section configured to attach to the first section.

12. Body compression device of claim 11, wherein the first section and the section attach to one another by utilizing one of adhesive fabric and a zipper.

13. Body compression device of claim 1, the pad forming a first section, the device additionally comprising a plurality of additional sections, wherein each of the additional sections is configured to attach to another of the additional sections or to the first section.

14. A body compression device for medical imaging of a patient, comprising:
a pad configured to wrap about the patient, the pad having first and second ends; and
at least one keeper to secure the first end of the pad to the second end of the pad about the patient;
such that adipose tissue of the patient is reshaped to facilitate the medical imaging when the pad is secured about the patient.

15. Body compression device of claim 14, the pad extending from armpits of the patient to thighs of the patient.

16. Body compression device of claim 14, the pad comprising material that is radiolucent, waterproof and flexible.

17. Body compression device of claim 14, the pad comprising radiation shielding.

18. A body compression device for medical imaging of a patient, comprising:
a dorsal panel;
a ventral panel;
elastic side panels affixed to one or both of the dorsal and ventral panels; and
means for securing the dorsal and ventral panels about the patient;
such that adipose tissue of the patient is reshaped to facilitate the medical imaging when the pad is secured about the patient.

19. Body compression device of claim 18 wherein the dorsal panel, the ventral panel and the side panels form a contiguous unit that wraps about the patient.

20. Body compression device of claim 18 wherein the dorsal panel and the ventral panel form separate sections.

21. Method of reducing total radiation exposure during CT imaging, comprising
positioning an X-ray fiducial device in proximity to a patient,
generating a CT pre-scan image of the patient,
determining a medical region of interest of the patient from a position of the device in the CT pre-scan image,
placing radiation shielding on at least one portion of the patient that is outside the medical region of interest, and
performing the CT imaging.

22. Method of claim 21, the step of positioning comprising securing a panel including the X-ray fiducial device to the patient, the step of determining comprising correlating information of the CT pre-scan image to a visual position of the device.

23. Method of claim 22, the step of determining further comprising utilizing visual markers that are at known distances from the fiducial device on the panel.

24. A method of medical imaging of a patient, comprising
positioning a substantially radiolucent body compression device adjacent to the patient,
securing the body compression device about the patient, thereby reshaping adipose tissue of the patient to reduce a horizontal diameter of the patient's cross-section, and
imaging the patient.

25. Method of claim 24 wherein the step of positioning comprises attaching a plurality of sections of the body compression device to one another.

26. Method of claim 24 wherein the step of imaging produces a satisfactory cross-sectional image of the patient with a lower X-ray dose than is required to produce a satisfactory cross-sectional image of the patient when the step of securing is not performed.

27. Method of claim 24 wherein the step of imaging comprises
performing a pre-scan,
evaluating a position of a radiopaque marker within an image formed by the pre-scan,
positioning radiation shielding on at least one portion of the patient in accordance with the position of the radiopaque marker within the image, and
performing cross-sectional imaging of the patient.
